(19) United States
(12) Patent Application Publication
(10) Pub. No.: US 2025/0375130 A1
(43) Pub. Date: Dec. 11, 2025

(54) METHODS AND SYSTEMS FOR ADMINISTRATION OF IN-VEHICLE INTOXICATION TESTING

(71) Applicant: Magna Electronics, LLC, Southfield, MI (US)

(72) Inventor: Caroline Chung, Royal Oak, MI (US)

(21) Appl. No.: 18/737,314

(22) Filed: Jun. 7, 2024

(51) Int. Cl. A61B 5/18; A61B 5/00

(52) U.S. Cl. CPC A61B 5/18; A61B 5/4845; A61B 5/4863; A61B 5/7435

(57) ABSTRACT

Methods and systems for administration of intoxication testing within a vehicle. In some implementations, the method may comprise displaying a fixed eye target on a graphical user interface of the vehicle and displaying a first prompt on the graphical user interface inducing the driver to perform a first movement sequence in which the driver is to stare at the fixed eye target while moving the driver's head in a first direction. A second prompt may be displayed on the graphical user interface inducing the driver to perform a second movement sequence in which the driver is to stare at the fixed eye target while moving the driver's head in a second direction. Received images of the driver's eyes may then be used to perform a nystagmus test and to assess whether the driver is intoxicated.

### SUMMARY

Current methodology for intoxication testing, such as the Drug Recognition Expert (DRE) procedure, requires a subject to move her eyes while her head is facing forward and steady. Nystagmus/intoxication testing in this manner is difficult to implement within a vehicle due to, for example, large arc that the subject would need to follow during the test, which makes implementation of the testing, as well as obtaining and processing test data, difficult, due to, for example, space limitations.

The present inventor has therefore determined that it would be desirable to provide systems and methods that overcome one or more of the foregoing limitations and/or other limitations of the prior art. Systems and methods for in-cabin intoxication testing, such as nystagmus testing, are therefore disclosed herein, which may provide a more convenient, efficient, and/or effective methodology for administration of intoxication testing within a vehicle.

Thus, in some embodiments and implementations, the inventive concepts disclosed herein may be used to facilitate administration of intoxication and/or nystagmus testing within a vehicle without requiring the driver/occupant/subject to follow/track a moving target during the testing.

In a more particular example of a computing device comprising a display within a vehicle, the computing device may be configured to display a first prompt to a test subject, such as the driver of the vehicle, indicating an initiation of an intoxication test. The first prompt may comprise a visual eye target for the subject/driver. The computing device may be further configured to display a second prompt to the subject/driver to turn the subject/driver's head in a first direction with the driver/subject's eyes fixed on the visual eye target.

The computing device may be further configured to display a third prompt to the subject/driver to turn the subject/driver's head in a second direction with the subject/driver's eyes fixed on the visual eye target. A status indication may be displayed to the subject/driver as to the status of the intoxication test.

In some embodiments, the visual eye target does not move during the intoxication test and may therefore be considered a fixed target.

In some embodiments, the status indication may comprise an indication about whether the driver has passed the intoxication test.

In some embodiments, the computing device may be further configured to display one or more error prompts indicative of one or more intoxication test errors. In some such embodiments, following the display of one or more error prompts, the computing device may be configured to repeat the initiation of the intoxication test.

In some embodiments, the computing device may be further configured to display an error prompt indicating a cause of one or more error prompts and/or instructing the driver how to repeat the intoxication test to avoid the one or more intoxication test errors. In some such embodiments, the computing device may further be configured to repeat the intoxication test, or a modified version thereof.

In some embodiments, the first prompt may comprise a textual instruction indicating that the driver should stare directly at the visual eye target during the intoxication test.

In some embodiments, the second prompt may comprise a dynamic graphical element that moves in the first direction to provide a visual cue to the driver to prompt movement of the driver's head in the first direction. In some embodiments, the third prompt may comprise a dynamic graphical element that moves in the second direction to provide a visual cue to the driver to prompt movement of the driver's head in the second direction.

In some such embodiments, the dynamic graphical element of the second prompt and the dynamic graphical element of the third prompt may each comprise a graphical element overlayed on the visual eye target that moves relative to the visual eye target in a direction and/or speed indicative of a preferred direction and/or speed of movement of the driver's head during the intoxication test.

In some embodiments, the dynamic graphical element may comprise an image of a human head.

In some embodiments, the visual eye target may be displayed at a fixed location. In some such embodiments, the fixed location may be at least substantially at a center of the dynamic graphical element.

In some embodiments, the computing device may be configured to display a first hold prompt in between the second prompt and the third prompt indicating a first time period during which the driver's head should be held steady before moving in the second direction. In some such embodiments, the computing device may be further configured to display a second hold prompt following the third prompt indicating a second time period during which the driver's head should be held steady following movement in the second direction.

In some embodiments, the first direction may be in an opposite direction relative to the second direction. For example, the first direction may be horizontally towards a first lateral side of the vehicle, and the second direction may be horizontally towards a second lateral side of the vehicle opposite the first lateral side of the vehicle. Alternatively, the first direction may be up and the second direction down.

In an example of a method for administration of an intoxication test sequence to a driver within a user interface of a vehicle, the method may comprise displaying a fixed eye target on a graphical user interface of the vehicle and displaying a first prompt on the graphical user interface, which may include text and/or imagery, inducing the driver to perform a first movement sequence in which the driver is to stare at the fixed eye target while moving the driver's head in a first direction. In some cases, the method may comprise receiving one or more images of the driver's eyes during the first movement sequence.

A second prompt may then be displayed on the graphical user interface inducing the driver to perform a second movement sequence in which the driver is to, once again, stare at the fixed eye target while moving the driver's head in a second direction. In some cases, the method may comprise receiving one or more images of the driver's eyes during the second movement sequence.

Received images of the driver's eyes may then be processed to perform a nystagmus test. The nystagmus test may be used to assess whether the driver is intoxicated.

In some implementations, the first prompt and/or the second prompt may comprise a dynamic graphical element that moves in a direction in which and/or a speed at which the driver is to move the driver's head during the first and/or second movement sequences.

In some implementations, the dynamic graphical element may pause between the first and second movement sequences for a predetermined time period to indicate that the driver should hold the driver's head steady while staring at the fixed eye target for the predetermined time period.

In some implementations, the dynamic graphical element may be overlayed on the fixed eye target. In some such implementations, the fixed eye target may be at least substantially centered within the dynamic graphical element. In some implementations, the dynamic graphical element may comprise an at least substantially spherical graphical element. In some such implementations, the dynamic graphical element may comprise another image, such as an image of a human head. In some such cases, the fixed eye target may comprise an eye of the human head, or both eyes of the human head.

In some implementations, the first prompt may further comprise text prompting the user to perform the first movement sequence and/or any of the additional movement sequences.

In an example of a system for intoxication testing within a vehicle according to some embodiments, the system may comprise an electromagnetic sensor, such as a camera, configured to receive electromagnetic signals from an occupant of the vehicle, such as the driver. The system may further comprise a graphical display configured to administer an intoxication test sequence to the occupant of the vehicle, wherein the intoxication test sequence comprises a series of prompts to the occupant. The system may further comprise an intoxication assessment module configured to assess potential intoxication of the occupant of the vehicle using electromagnetic signals received by the camera or other electromagnetic sensor during the intoxication text sequence.

In some implementations, the graphical display may comprise a graphical user interface.

In some implementations, the intoxication assessment module may comprise a nystagmus detection module, and the intoxication test sequence may comprise a nystagmus test sequence.

In some such implementations, the nystagmus test sequence may comprise displaying a first prompt on the graphical display inducing the occupant to perform a first movement sequence in which the occupant is to stare at a fixed eye target displayed on the graphical display while moving the occupant's head in a first direction. In some such implementations, the nystagmus test sequence may further comprise displaying a second prompt on the graphical display inducing the occupant to perform a second movement sequence in which the occupant is to stare at the fixed eye target while moving the occupant's head in a second direction.

In some implementations, the electromagnetic sensor may comprise a camera. In some such implementations, the camera may be configured to receive a series of images of at least one of the occupant's eyes during the intoxication test sequence.

Some implementations may further comprise an alcohol sensor, such as a breath sensor, a passive alcohol sensor, and/or a touch sensor, configured to detect an intoxicant and/or alcohol level from the breath of an occupant of a vehicle, the ambient air in the vehicle, and/or the blood (indirectly) of the occupant.

In some implementations, the alcohol sensor may be positioned within the steering wheel of the vehicle. In some such implementations, the system may be configured to initiate the intoxication text sequence upon detecting a threshold intoxicant and/or alcohol level using a breath sensor or other alcohol sensor.

In some implementations, the intoxication test sequence may comprise a nystagmus test sequence.

In some implementations, the series of prompts to the occupant may comprise at least one prompt instructing the occupant to stare at an object displayed on the graphical display (preferably a fixed target) while moving the occupant's head. In some implementations, the series of prompts to the occupant may comprise at least one prompt instructing the occupant to stare at the object while moving the occupant's head laterally.

The features, structures, steps, or characteristics disclosed herein in connection with one embodiment may be combined in any suitable manner in one or more alternative embodiments.

### BRIEF DESCRIPTION OF THE DRAWINGS

Non-limiting and non-exhaustive embodiments of the disclosure are described, including various embodiments of the disclosure with reference to the figures, in which:

FIG. 1 depicts a steering wheel and graphical display, such as a graphical user interface, for use in connection with a system for in-vehicle intoxication testing according to some embodiments;

FIG. 2 depicts some of the elements of a system for in-vehicle intoxication testing according to alternative embodiments;

FIGS. 3A-3F depicts various examples of screenshots of an in-vehicle graphical display, such as a graphical user interface, that may be used to direct a driver or other vehicle occupant to perform a nystagmus test;

FIG. 4 depicts a system for administration of in-vehicle intoxication testing according to some embodiments; and

FIGS. 5A and 5B are flowcharts depicting an example of a method for administration of in-vehicle intoxication testing according to some implementations.

### DETAILED DESCRIPTION

It will be readily understood that the components of the present disclosure, as generally described and illustrated in the drawings herein, could be arranged and designed in a wide variety of different configurations. Thus, the following more detailed description of the embodiments of the apparatus is not intended to limit the scope of the disclosure but is merely representative of possible embodiments of the disclosure. In some cases, well-known structures, materials, or operations are not shown or described in detail.

As used herein, the term “substantially” refers to the complete or nearly complete extent or degree of an action, characteristic, property, state, structure, item, or result to function as indicated. For example, an object that is “substantially” cylindrical or “substantially” perpendicular would mean that the object/feature is either cylindrical/perpendicular or nearly cylindrical/perpendicular so as to result in the same or nearly the same function. The exact allowable degree of deviation provided by this term may depend on the specific context. The use of “substantially” is equally applicable when used in a negative connotation to refer to the complete or near complete lack of an action, characteristic, property, state, structure, item, or result. For example, structure which is “substantially free of” a bottom would either completely lack a bottom or so nearly completely lack a bottom that the effect would be effectively the same as if it completely lacked a bottom.

Similarly, as used herein, the term “about” is used to provide flexibility to a numerical range endpoint by providing that a given value may be “a little above” or “a little below” the endpoint while still accomplishing the function associated with the range.

The embodiments of the disclosure may be best understood by reference to the drawings, wherein like parts may be designated by like numerals. It will be readily understood that the components of the disclosed embodiments, as generally described and illustrated in the figures herein, could be arranged and designed in a wide variety of different configurations. Thus, the following detailed description of the embodiments of the apparatus and methods of the disclosure is not intended to limit the scope of the disclosure, as claimed, but is merely representative of possible embodiments of the disclosure. In addition, the steps of a method do not necessarily need to be executed in any specific order, or even sequentially, nor need the steps be executed only once, unless otherwise specified. Additional details regarding certain preferred embodiments and implementations will now be described in greater detail with reference to the accompanying drawings.

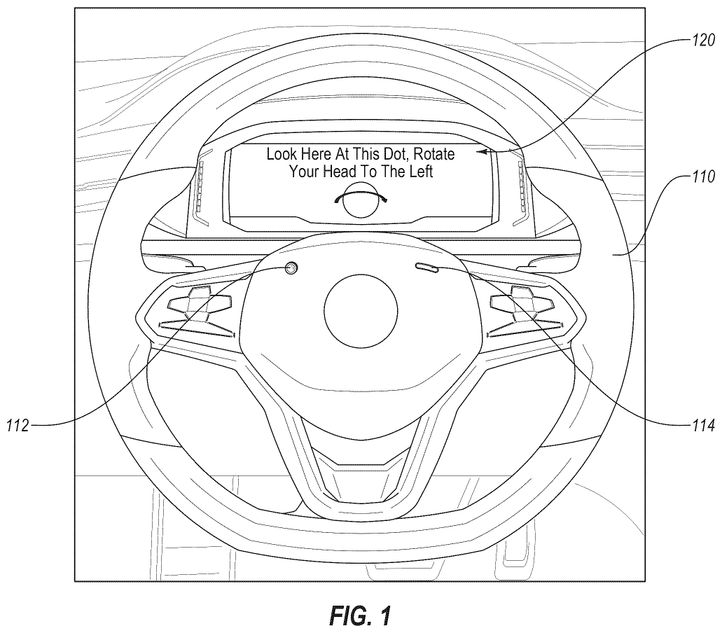

FIG. 1 depicts certain physical components of a system for administering intoxication testing to an occupant, such as a driver, within the cabin of a vehicle. As shown in this figure, one or more sensors may be positioned at various locations within the cabin of the vehicle to facilitate performing intoxication testing. For example, the steering wheel 110 shown in FIG. 1 comprises a camera 112 and an intoxication sensor 114.

Camera 112 may be used to capture images of the driver and/or other occupants of the vehicle. In some embodiments, camera 112 may be directed to the driver's face and/or configured to capture images, either still or moving, of the driver's eyes. Thus, in some embodiments, camera 112 may be configured with a field of view that is preset to be directed towards the area where all or most driver's heads/faces would be located during driving. In some embodiments, the field of view may be adjustable to allow for targeting unusually short or tall drivers, or to zoom in on a particular driver or other occupant's eyes, for example, however.

Of course, although the example of FIG. 1 comprises a camera 112 that is located on the steering wheel 110 and is therefore directed towards a driver, it should be understood that, in other embodiments, camera 112 may be located elsewhere and/or may comprise a field of view that is directed at other seats in the vehicle, including the passenger seat and rear seats within the vehicle. In addition, it should be understood that additional cameras may be located throughout the cabin of the vehicle as desired for this purpose—i.e., to allow for performing intoxication testing, as described herein, on other vehicle occupants. In addition, although the discussion herein will tend to focus on the use of a camera for certain testing procedures, it also should be understood that other sensors, including other electromagnetic sensors, such as RADAR sensors, may be used instead of, or in addition to, camera 112. Element 112 may therefore alternatively be referred to herein as an electromagnetic sensor or, even more generally, as a sensor, herein.

The intoxication sensor 114, which, as shown in FIG. 1, may also be positioned on the steering wheel 110 in some preferred embodiments, may be configured as an alcohol sensor. As such, sensor 114 may be configured to receive, either passively or actively, air from within the cabin and measure the concentration of alcohol in the sampled air. In some embodiments, sensor 114 may comprise a passive alcohol sensor, which may comprise a fuel cell, electrodes, semiconductor technology, MEMS technology, or any other technology for detecting alcohol concentrations available to those of ordinary skill in the art.

In other embodiments, sensor 114 may comprise a breath sensor or breathalyzer, which may be configured to determine a blood alcohol concentration (“BAC”) measurement by a driver/user intentionally exhaling into the sensor. In such embodiments, sensor 114 may comprise a retractable tube and/or mouthpiece to ensure that the user exhales directly into the sensor 114.

In still other embodiments, sensor 114 may comprise a touch sensor. In some such embodiments, sensor 114 may be configured to use, for example, infrared radiation to measure alcohol levels in a driver/user's blood via the skin, such as on a fingertip. Although placement on the steering wheel 110 may still be feasible and/or desirable for such sensors, they may also be placed elsewhere, such as on a gear shift, next to a graphical display and/or graphical user interface, or on the dash, for example.

The system of FIG. 1 further comprises a graphical display 120. Graphical display 120 may, as discussed in greater detail below, be configured to direct the occupant/driver to perform another intoxication test procedure, which may be in lieu of a test using sensor 114, or may be in addition to or as an alternative to such an intoxication test. For example, in some implementations of intoxication system testing, an initial result may be obtained using sensor 114, which may then trigger a secondary test using, in part, display 120 to confirm the results of the initial test. Alternatively, intoxication testing using graphical display 120 may be performed first, or without use of an alcohol sensor 114 at all.

In some embodiments, graphical display 120 may be configured to display a series of instructions, which may include images and/or moving images, to administer a nystagmus intoxication test. In some embodiments, graphical display 120 may therefore be passive—i.e., it may only be configured to display such instructions/images to the user without allowing the user to respond/input data. Alternatively, however, in some preferred embodiments, display 120 may comprise a user interface 120. In other words, display 120 may allow a user/occupant to interact with the intoxication system and/or other systems of the vehicle.

As shown in FIG. 1, an initial step of the administration of a nystagmus intoxication test may instruct the user to stare at a particular location and/or image in the display 120, such as a fixed dot, circle, or other fixed target. The instruction may further instruct, either in the same image/instruction or a subsequent image/instruction, the user to turn her head while continuing to stare at the dot/circle/target, which may make the nystagmus test easier to conduct and/or process than a test in which the user turns her head and eyes while tracking a moving object/target with her eyes.

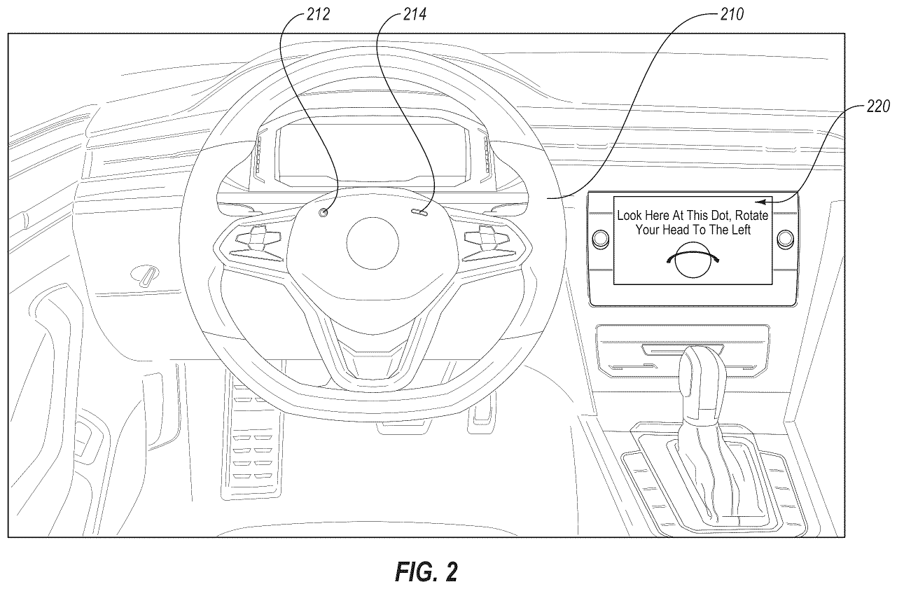

Another example of a system for administering intoxication testing to an occupant, such as a driver, within the cabin of a vehicle is shown in FIG. 2. As shown in this figure, a steering wheel 210 is provided with one or more sensors to facilitate intoxication testing. In the depicted embodiment, a camera 212 is provided, along with an alcohol sensor 214 for detection of alcohol in the cabin, the driver/occupant's breath, and/or the blood alcohol concentration (BAC) of the driver/occupant's blood.

As mentioned above, camera 212 may be positioned at any desired location within the vehicle, preferably to target the driver/occupant's face, and even more particularly the eyes. Thus, although the face of the steering wheel 210 may be a desirable location for doing this, other locations may be suitable as well, such as on the rear-review mirror, on the dashboard, and within the windshield. In addition, other electromagnetic sensors, such as RADAR sensors, may be used, either in addition to or as an alternative to a camera.

As also mentioned above, sensor 214 may comprise any of a number of different sensors configured to provide a blood alcohol concentration of a driver/occupant or otherwise provide an indication of the presence and/or concentration of alcohol in the air in the cabin and/or the breath of the driver/occupant. Examples of such sensors include, but are not limited to, passive alcohol sensors, breath sensors or breathalyzers, and touch sensors.

In addition, although it may be convenient to locate one or more alcohol sensors 214 on the steering wheel, as shown in FIG. 2, it should be understood that this is but an example. In other embodiments, one or more sensors 214 may be located elsewhere within the vehicle, such as on a gear shift, next to a graphical display and/or graphical user interface, or on the dashboard, for example.

Again, a graphical display 220 is provided, which may comprise a graphical user interface in some embodiments. In the system of FIG. 2, display 220 is provided on a center console or instrument panel located adjacent to the gear shift rather than on the dashboard. Once again, this display 220 may be used to initiate and prompt a driver/user through a series of steps involved in a nystagmus test. In preferred embodiments and implementations, this test is conducted so that the driver/user can focus on a fixed point that may be displayed on the graphical display 220 while turning her head, in some cases in both directions. As explained in greater detail below, in some embodiments, additional steps may be involved in the prompts/testing, such as holding the head steady for a fixed time period while continuing to stare at the fixed point.

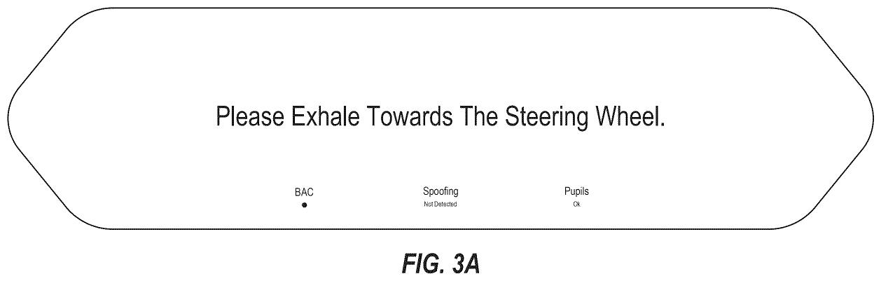

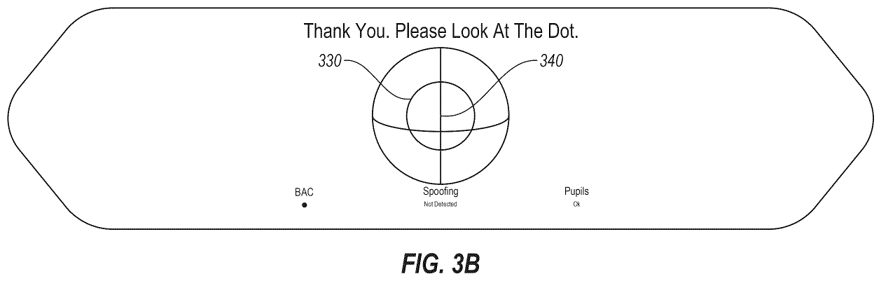

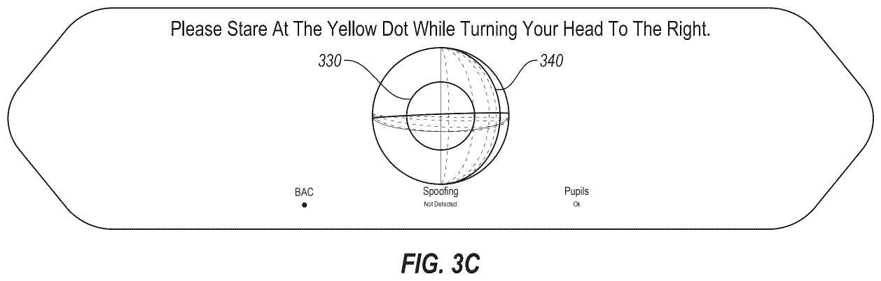

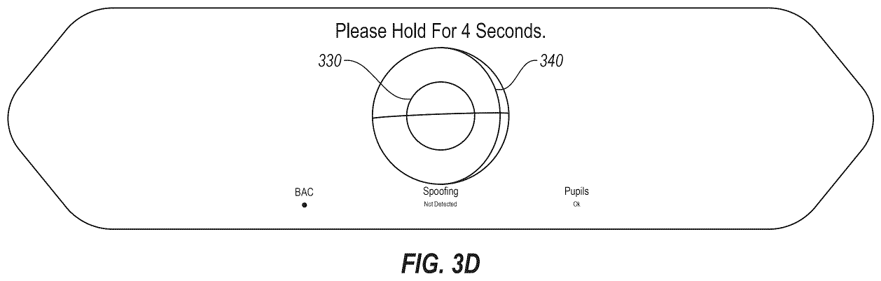

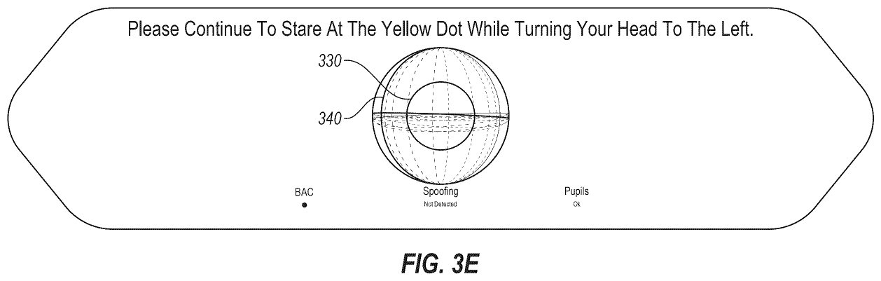

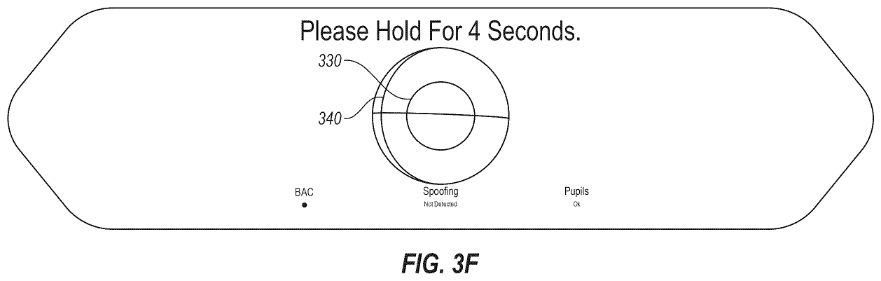

FIGS. 3A-3F depict a series of screenshots from a user interface and/or graphical display within a vehicle according to some embodiments. The instructions and features depicted on these screenshots may be displayed in sequence, in some cases with sounds and/or video/moving imagery, to walk a driver/occupant through a test sequence for detecting intoxication. In some cases, the instructions may only be given audibly, or optionally by the user only audibly. In the example depicted, this test sequence is specifically designed as a nystagmus test and, even more particularly, a fixed eye target nystagmus test in which a user maintains a fixed stare/gaze at a target while moving her head to the side, side to side, up, down, or up and down.

FIG. 3A depicts an initial instruction in an intoxication test sequence. In this step, a user is instructed to exhale towards the steering wheel. This instruction therefore assumes the presence of a sensor for detecting the presence of alcohol on the steering wheel. It should therefore be understood that the screenshot of FIG. 3A, along with each of the others, is but an example and may be removed and/or replaced with similar messages/displays. For example, as mentioned above, the steering wheel need not have an alcohol sensor in all contemplated embodiments. Moreover, even if such a sensor is present, it need not rely on the driver/occupant exhaling intentionally towards the steering wheel, or even exhaling intentionally at all. Similarly, in some embodiments and implementations, a nystagmus test may be directed by the vehicle system/display without use of any alcohol sensors. However, in preferred embodiments, at least a camera is used with such testing, as should be apparent after a complete review of the contents of this disclosure.

Other optional features are also shown on the screenshots of FIG. 3A, such as a BAC indicator. In some embodiments and implementations, this may display an approximate blood alcohol concentration that has been detected by an in-cabin intoxication detection system, such as one derived from a passive alcohol sensor or breathalyzer sensor. In some cases, the BAC portion of the screen may simply be a binary indicator, which may indicate the presence of alcohol being detected using, for example, a checkmark or the dot shown in the figure, with the absence of a mark indicating that no alcohol has been detected in the breath, cabin, and/or blood of the driver/occupant.

Next to the BAC indicator is a “Spoofing” indicator. As discussed below, some embodiments may comprise features to detect whether a driver/occupant is attempting to “spoof” the intoxication testing by avoiding detection of alcohol. This may be done, for example, by use of filters in the system, by using occupants other than the driver to perform a breathalyzer, or by being too far from, or too close to, the relevant camera and/or other sensors. In some such embodiments and implementations, the display/user interface may provide an indication of whether such spoofing has been detected or suspected, which may also result in restarting the testing, a test failure result, notifications, and/or other corrective actions and/or instructions.

Another indicator is provided in the depicted example next to the spoofing indicator, which provides an indication of whether the pupils can be adequately detected from the driver/occupant's current position. In the depicted example, this is another binary indicator (OK), but other embodiments are contemplated. For example, in some embodiments and implementations, in instances in which the pupils cannot be seen or cannot detected with sufficient detail to perform the test, the display may provide another instruction, either on the same display screen or a subsequent one, to instruct the user what must be done in order to improve the visualization of the pupils. For example, the system may display an instruction to “remove glasses,” “sit closer,” lean forward,” “look towards the screen [or other camera location],” etc.

FIG. 3B provides another screenshot of an administration of an in-cabin nystagmus test sequence. The screenshot of FIG. 3B may follow that of FIG. 3A, assuming the initial intoxication test has been passed. Alternatively, a modified version of the screenshot of FIG. 3B (removing the “Thank You,” for example) may be considered the first step in the test sequence in some cases.

As indicated in FIG. 3B, a fixed target, such as a dot 330, is provided on the display. The dot 330 provides a fixed target for the driver/occupant to stare at during the test. Thus, preferably, the display also includes instructions for the driver/occupant to look/stare at the fixed target/dot 330. Although, as shown in the figure, textual instructions may be provided, alternatively or additionally, audible instructions may be provided via, for example, the speakers in the vehicle. Although a dot 330 or circle may be considered a simple example of a fixed target, any number of fixed targets may be used, such as an image of an eye or any other desired icon and/or image. In some cases, multiple fixed targets may be provided, such as two fixed images of eyes.

In the depicted embodiment, a dynamic graphical element 340 surrounds the fixed target 330. As will be apparent after considering the other screenshots in the sequence of FIGS. 3A-3F, dynamic graphical element 340 may move about the fixed target 330 to assist the driver/occupant in performing the nystagmus test during the test. In the depicted embodiment, dynamic graphical element 340 comprises a circle or spherical element that surrounds, or appears to surround, the fixed target 330. Although the depicted example is rather simple, in that both of the graphical elements are essentially circles, with the dynamic element 340 appearing to be spherical so as to be movable about the fixed target 330, once again, this is but an example. A wide variety of alternative dynamic elements are contemplated and/or would be apparent to those of ordinary skill in the art after having received the benefit of this disclosure.

For example, in some embodiments and implementations, the dynamic element 340 may comprise the shape of a human head or part of a human head. This may allow the head to move as needed so as to provide a model for the driver/occupant to move her head accordingly. In some such cases, the dynamic element or elements (such as eyes within the aforementioned head image) may have a transparent look so as to allow for a fixed target to remain stationary with respect to the dynamic element. Alternatively, however, the fixed target may be positioned on part of the dynamic element or may be positioned adjacent to but separate from the dynamic element.

As shown in FIG. 3C, text and/or audio may then instruct the user to move her head while continuing to look/stare at the fixed target 330. In the depicted example, the instruction indicates turning the head to the right. However, this is but an example. The instructions may alternatively first instruct the user to turn her head to the left, upwards, or downwards. As this instruction is displayed and/or audibly played, the dynamic graphical element 340 moves in the direction of the instruction—i.e., to the right in the depicted example. A series of dashed lines are included in FIG. 3C to indicate movement of the center/vertical line of the dynamic graphical element 340 to the right. Thus, technically, FIG. 3C should not be considered a screenshot, but rather to depict and encompass a series of screenshots indicating this movement.

After the dynamic graphical element 340 has moved all the way in one direction (to the right in the example), instructions may be provided to hold the user's head in place, as indicated in FIG. 3D, for a fixed time period, such as three seconds. In some cases, additional instructions may be provided to keep the user's head steady and/or to continue to stare at the fixed target 330. As discussed in greater detail below, a sensor, such as a camera, may capture images of the user's eyes and/or pupils during this fixed stage of the test (as with other steps) to evaluate signs of nystagmus and/or intoxication.

Following the fixed time period, the dynamic graphical element 340 of the display may move in another direction, such as an opposite direction of the initial movement. In the depicted example, this movement is to the left, as shown in FIG. 3E. Instructions may also be provided to the user to move her head in this direction, preferably tracking the movement of the dynamic graphical element 340 while keeping a steady gaze on the fixed target 330.

After the dynamic graphical element 340 has moved all the way in the second and/or opposite direction as the initial movement (to the left in the example), instructions may be provided to hold the user's head in place, as indicated in FIG. 3F, for another fixed time period, which may but need not be the same as the first fixed time period. In some cases, additional instructions may be provided to keep the user's head steady and/or to continue to stare at the fixed target 330.

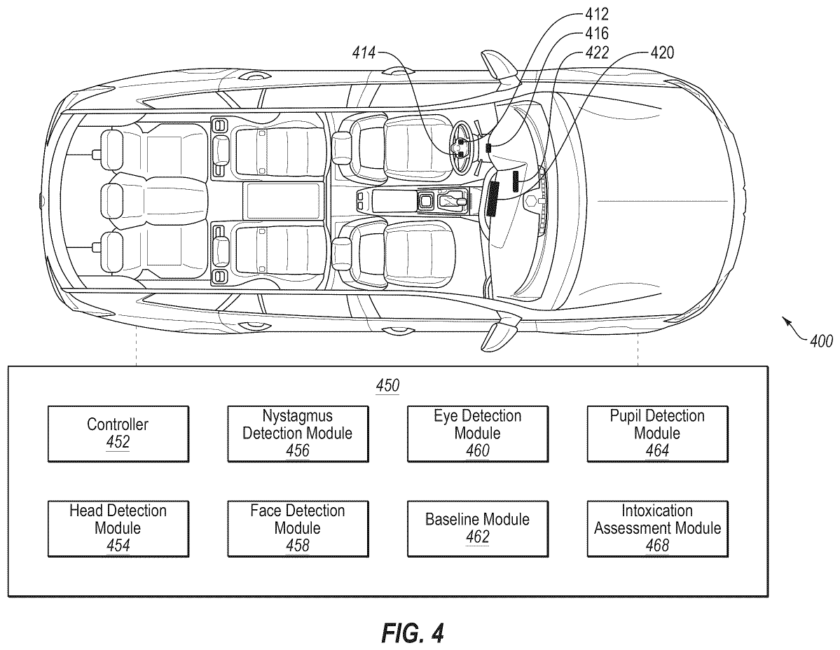

FIG. 4 illustrates an example of a vehicle 400 comprising a system for administration of an intoxication test within a vehicle according to some embodiments. The vehicle shown in FIG. 4 comprises a camera 412, which may be positioned at any desired location within the vehicle, but preferably is positioned in a convenient location to view and target the driver/occupant's face, and even more particularly, the eyes. Thus, although the camera 412 is shown on the steering wheel of the vehicle, a variety of alternative locations may be suitable, such as on the rear-review mirror, on or in the dashboard, within the windshield, etc. In addition, although only one camera is shown, any number of cameras may be used, as desired. Similarly, other sensors, such as RADAR sensors, may be used, either in addition to or as an alternative to cameras.

Indeed, vehicle 400 comprises a second sensor 422 positioned on the dashboard above the steering wheel. Sensor 422 may comprise, for example, a RADAR sensor, a LIDAR sensor, or a second camera. RADAR sensor 422 may be configured to send RADAR signals towards the driver and/or other occupant(s) and receive signals reflected back to the RADAR sensor 422. Processing of signals from the RADAR sensor 422 may then be used to determine one or more parameters of the driver/occupant, such as, for example, heart rate, breathing rate, or other vital sign rate, movement patterns, size/shapes, etc. Such signals may, in some embodiments, be used to detect intoxication and/or detect “spoofing”—i.e., attempts to avoid accurate detection of alcohol and/or intoxication.

Vehicle 400 further comprises an alcohol sensor 414 for detection of alcohol in the cabin, the driver/occupant's breath, and/or the blood alcohol concentration (BAC) of the driver/occupant's blood. Again, sensor 414 may comprise any of a number of different sensors configured to provide a blood alcohol concentration of a driver/occupant or otherwise provide an indication of the presence and/or concentration of alcohol in the air in the cabin and/or the breath of the driver/occupant such as, for example, passive alcohol sensors, breath sensors or breathalyzers, and touch sensors.

As previously discussed, although it may be convenient to locate one or more alcohol sensors on the steering wheel, as shown in FIG. 4, it should be understood that, like the camera 412, sensor 414 may be located elsewhere within the vehicle, such as on a gear shift, next to a graphical display and/or graphical user interface, or on the dashboard, for example. Similarly, multiple alcohol and/or intoxication sensors may be provided in vehicle 400 as desired.

Vehicle 400 further comprises a graphical display 420, which may comprise a graphical user interface in some embodiments. In the system of FIG. 4, display 420 is provided on a center console or instrument panel located adjacent to the gear shift, but this is merely an example. Display 420 may be located at any convenient and/or desired location within the vehicle. As previously mentioned, display 420 may be used to initiate and prompt a driver/user through a series of steps involved in a nystagmus test. In preferred embodiments and implementations, this test is conducted so that the driver/user can focus on a fixed point that may be displayed on the graphical display 420 while turning her head, in some cases in two opposite directions, such as side to side or up and down.

In some embodiments, a lighting element 416 and/or light sensor may also be provided. This element may be used to ensure that sufficient light is directed to the driver/occupant's face and/or eyes during the test. Similarly, lighting element 416 may be used to intentionally change the size of the test subject's pupils, which may be needed in situations in which initial test results are inconclusive. In some cases, element 416 may further comprise a light sensor configured to determine whether the ambient lighting is sufficient to perform the test without additional light.

In addition to the aforementioned hardware elements, a system 450 may be provided within the vehicle for execution of various software, hardware, and/or firmware elements configured to gather data, process data, and/or perform various steps within vehicle 400. System 450 may comprise a controller 452, which may be configured to process data from the various sensors/sensor modules 412/414/416/422. As used herein, the term “controller” refers to a hardware device that includes a processor and preferably also includes a memory element. The memory may be configured to store one or more of the modules referred to herein and the controller 452 and/or one or more processors may be configured to execute the modules to perform one or more processes described herein.

System 450 further comprises a head detection module 454 that is communicatively and/or operatively coupled with one or more (or all) of the sensors/sensor modules 412/414/416/422, preferably at least being so coupled with the camera 412. Of course, in some embodiments, a separate detection module may be provided for each sensor and/or sensor module, if desired.

Each of the various modules disclosed herein, including head detection module 454, may be configured to receive raw, sensed data from the various sensors/sensor modules and process this data to achieve a desired outcome. For example, head detection module 454 may be configured to receive image data from the camera sensor 412 and/or radar sensor 422. The head detection module 454 may detect the head of the driver, or another occupant in the vehicle 400, in the image data using a head detection algorithm, such as a Haar cascade, a convolutional neural network (CNN), or in any other manner available to those of ordinary skill in the art.

In some embodiments, the head detection module 454 may determine a region of interest (ROI) or the like including the head of the driver or other desired occupant in the image. The head detection module 454 may transmit the ROI of the image data to a face detection module 458. The face detection module 458 may then detect the face of the driver in the ROI including the head. More specifically, the face detection module 458 may determine a second ROI including the face of the driver from the first ROI including the head. The face detection module 458 may detect the face using a face detection algorithm, such as using feature extraction and/or constraints.

The head detection module 454 may, in some embodiments, also determine a pose of the head of the driver in the image. The pose may be, for example, a mesh connecting key points of the head of the driver. The head detection module 454 may determine the pose of the head, for example, using a pose detection algorithm for human heads.

The face detection module 458 may transmit another ROI of the image data including the face to an eye detection module 460. The eye detection module 460 may be configured to detect one or more of the eyes of the driver or other vehicle occupant in the ROI including the face. The eye detection module 460 may be configured to determine one or more ROIs including the eyes of the driver in the ROI including the face from the image data. The eye detection module 460 may detect the eyes using an eye detection algorithm using feature extraction and/or constraints.

The eye detection module 460 may be configured, in turn, to specifically detect pupils of the eyes of the driver or other occupant from the image data using, for example, a pupil detection algorithm and/or pupil detection module 464. The eye detection module 460 and/or pupil detection module 464 may be configured to determine one or more characteristics of the pupils of the eyes of the driver and/or other occupant, such as pupil size/diameter, location of a center of the pupil(s), pupil movement, velocity, and/or acceleration characteristics, and the like. This data may be used by other modules, such as the nystagmus detection module 456, to assess the presence of nystagmus and/or intoxication.

The nystagmus detection module 456 may be configured to output a series of instructions to a display screen and/or graphical user interface, as previously mentioned, and may be configured to coordinate the operation of various hardware elements, such as camera 412 and/or lighting element 416, to ensure that the data needed in order to evaluate the results of such testing are reliably obtained.

In addition, the nystagmus detection module 456 may be configured to process the incoming image data to evaluate the presence, or the likelihood of the presence, of nystagmus and/or intoxication. For example, when a pupil and/or a center of a pupil moves by more than a predetermined amount during a predetermined time period, and/or moves by more than a predetermined amount at least a predetermined number of times (e.g., two or more) between predetermined times, such as between consecutive images, an indication may be made of nystagmus, or an indication that is used in a final processing algorithm to determine nystagmus.

Similarly, during portions of a test sequence when a user is instructed to hold her head steady, other data processing steps may be implemented, such as determining the distance and/or frequency of movement of the pupils during these stages. Again, threshold amounts may be used to trigger a nystagmus fail, or may be used in a larger score at the end of the test sequence that may increase, or decrease, the chances of a nystagmus fail, such as becoming part of a larger, final score of the testing to determine a pass/fail, and/or to determine a score indicative of a likelihood of intoxication.

A baseline module 462 may also be included in some embodiments, which baseline module 462 may be configured to provide a baseline for the testing from previously obtained data to make the testing and/or processing more efficient and/or accurate. For example, in some embodiments, baseline module 462 may be configured to allow a user to select from a series of profiles for drivers of the vehicle, and/or may automatically attempt to match the driver to such a profile using previously stored image data of various faces of the drivers having saved profiles. By matching the driver's face using image data from camera 412, a face that most closely matches the face of the driver captured in the image data from the camera 412 may be loaded for the present driver.

In some cases, the baseline module 462 may use other data stored from such a profile for the driver, such as heart rate or other vital sign data, body pose data, baseline pupil size data, and/or baseline facial pose data. Additional data for any of these parameters may be obtained by the system 450 and compared with the baseline data for the relevant driver. This comparison may then be fed to intoxication assessment module 468 for further processing. Examples of systems and methods for detecting vital sign data using RADAR signals can be found in U.S. patent application Ser. No. 18/072,662 titled SYSTEMS AND METHODS FOR VEHICLE OCCUPANT VITAL SIGN DETECTION, which was filed on Nov. 30, 2022, and which is hereby incorporated by reference in its entirety.

For example, such further processing by intoxication assessment module 468 may comprise applying one or more thresholds and/or other predetermined conditions for triggering one or more actions, such as triggering an indication of intoxication. In some cases, the intoxication assessment module 468 may consider whether the heart rate of the driver is greater than the baseline heart rate of the driver by at least a predetermined amount, whether the breathing rate of the driver is greater than the baseline breathing rate of the driver by at least a predetermined amount, whether a body and/or facial pose of the driver differs from respective baseline poses of the driver by at least a predetermined amount, etc. Similarly, a pupil size of the driver may be evaluated and compared with a baseline pupil size for the same driver to determine whether they differ by a predetermined amount. In some cases, there may be more than one predetermined amount, with the size of the difference being indicative of a greater likelihood of intoxication.

In some cases, the intoxication assessment module 468 may also, or alternatively, be configured to receive more definitive data from other sensors, such as a passive alcohol sensor, touch sensor, breath sensor, or other alcohol sensor 414. This data may be used, either alone or in conjunction with other data, such as data from baseline module 462 and/or nystagmus detection module 456, to determine whether the driver/occupant is intoxicated, how likely intoxication is, and/or how intoxicated/impaired the driver occupant is.

Additional details of intoxication detection and/or confirmation are provided in U.S. patent application Ser. No. 18/117,081 titled MULTI-SENSOR INTOXICATION DETECTION, along with U.S. patent application Ser. No. 18/117,102 titled “SYSTEMS AND METHODS FOR CONFIRMATION OF INTOXICATION DETERMINATION,” both of which were filed on Mar. 3, 2023, and both of which are hereby incorporated by reference in their entireties.

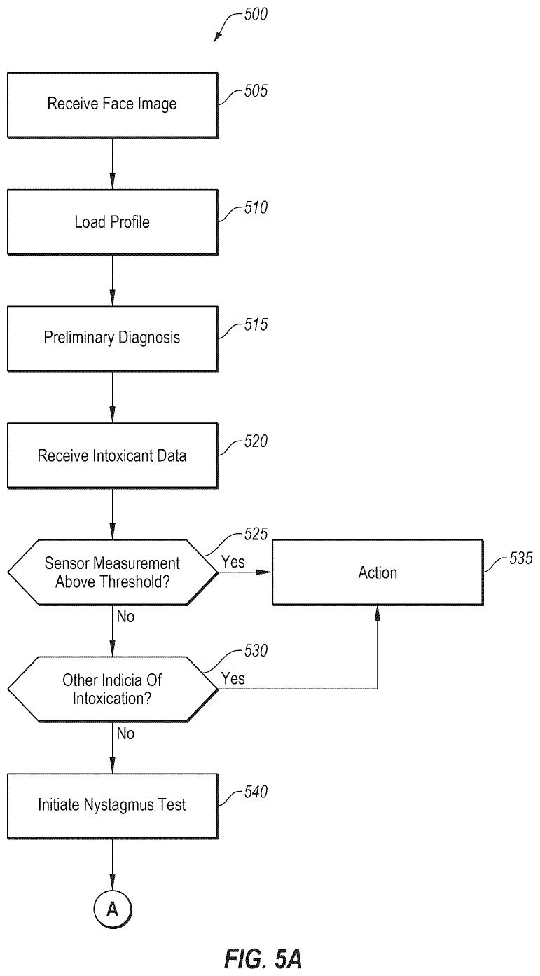

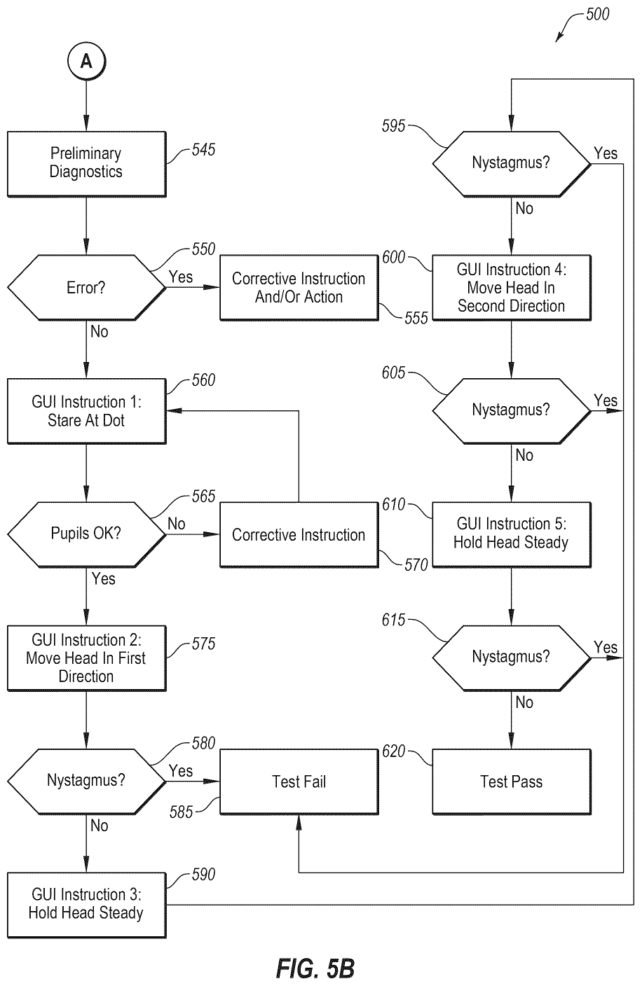

FIGS. 5A and 5B provide a flow chart illustrating an example of a method 500 for performing an in-cabin intoxication test according to some implementations. Method 500 may begin at 505 at which point image data is received from one or more sensors, such as one or more cameras, within the vehicle. In some implementations, this image data may comprise facial image data, as indicated in FIG. 5A. However, it should be understood that this data may be more comprehensive in some cases and may therefore include data from the body and/or other data from other sensors, such as radar sensor data indicative of vital sign rates, for example.

This data may be compared to stored data associated with one or more profiles, which may comprise data from other drivers of the vehicle and may each comprise a series of data stores, including, for example, along with facial data, baseline data, such as baseline heartrates, baseline pupil size, baseline body and/or face poses, etc. If a match of a stored profile is found, the profile may be loaded at 510. Of course, some embodiments need not have stored profiles and may therefore skip step 510.

In some cases, a preliminary diagnosis and/or score may then be made at 515. For example, if, despite a match being found using image data for the current driver, the baseline data, such as baseline heart rate, baseline face and/or body pose, and/or baseline pupil size, varies from the data that may have been acquired from the current image data by a predetermined amount, an initial assessment and/or score may be made at 515 as to intoxication or the likelihood of intoxication. This data may be stored and used later after receiving more detailed information to obtain a full score and/or test results.

Intoxicant data may then be received at 520. This data may be obtained from one or more alcohol sensors, such as sensor 414 in FIG. 4, which may be passive sensors, touch sensors, or breath sensors. A determination may then be made at 525 as to whether the intoxicant data exceeds a threshold. For example, in vehicles having a breath sensor, step 515 may comprise detecting whether the BAC is above a legal limit in the car's current location. Alternatively, in some cases, the threshold may be any detectable amount. For example, if the driver is below the legal drinking age, the threshold may be zero, or close to zero. Similarly, in some embodiments including passive alcohol sensors, step 525 may be to determine whether the passive sensor has detected the presence of any alcohol in the cabin. The detection of any alcohol within the cabin and/or driver may, in some cases, be used as a trigger to initiate further testing, such as nystagmus testing.

If there is a measurement of alcohol, either at all or above a predetermined threshold, method 500 may proceed to step 535 at which point an action may be taken. For example, in some implementations, the vehicle may be shut down, prevented from starting, and/or prevented from moving. If step 535 occurs while the vehicle is moving, control of the vehicle may be overtaken (if the vehicle is autonomous or semi-autonomous) and/or automatically slowed to a stop. In some cases, an electronic signal indicating that the driver is intoxicated may be sent to a remote device. Alternatively, or additionally, step 535 may comprise displaying and/or audibly playing an indicator that the driver is intoxicated via, for example, a light, a message, and/or a speaker.

In addition, in some cases, step 535 may instead trigger testing for other indicia of intoxication, as indicated at step 530, or initiation of a nystagmus test, as indicated at step 540. In other words, in some implementations, an indication of the presence of alcohol, alcohol above a threshold value, and/or a BAC above a threshold value may be used to trigger a nystagmus test rather than using a nystagmus test as a back up to the initial testing, as indicated in FIG. 5A.

At step 530, one or more indicia of intoxication other than those obtained by direct sensor measurement in previous steps may be tested. In some cases, these tests may rely on cameras and/or radar sensors to assess, for example, vital signs, body pose conditions, head pose conditions, and/or facial conditions to assess signs of intoxication. For example, if the breathing rate and/or heart rate of the driver exceeds that of a threshold, which may be a baseline rate for the particular profile/driver being tested, this may trigger an indicia of intoxication. The predetermined amount may be, for example, above about 10 percent of the baseline heart rate or another vital sign rate value.

As another example, which may be in addition to or as an alternative to vital sign testing, step 530 may comprise determining whether the body pose of the driver differs from a standard, such as whether it differs from a baseline body pose of the driver, by at least a predetermined difference. This may indicate that the driver is, for example, slouching relative to his or her normal posture. The predetermined difference may be, for example, a predetermined distance per keypoint or another suitable metric.

As another example, which may be in addition to or as an alternative to vital sign testing, step 530 may comprise determining whether the facial pose and/or condition of the driver differs from a standard, such as whether it differs from a baseline facial pose and/or condition of the driver, by at least a predetermined difference. This may indicate that the driver's face is, for example, droopy (relaxed muscle tone) relative to his or her normal pose. The predetermined difference may be, for example, a predetermined distance per keypoint or another suitable metric.

As another example, which may be in addition to or as an alternative to vital sign testing, step 530 may comprise determining whether the pupil diameter of the driver differs from a standard, such as whether it differs from a baseline pupil size of the driver, by at least a predetermined value. The predetermined may be, for example, approximately 10 percent of the baseline diameter or another suitable value. In various implementations, the predetermined value may be a distance.

At step 540, an initial nystagmus test may be initiated. Again, this test may be a test to confirm lack of intoxication that has been suggested by other tests or may be a test to confirm intoxication that has been suggested by other tests. In other cases, this test may take place without use of any other alcohol or intoxication testing, however. In other words, some systems may be configured to rely solely on nystagmus testing for intoxication testing without use of any additional steps and/or sensors.

Step 540 may comprise, for example, displaying an initial instruction and/or sequence of instructions on a display and/or graphical user interface, such as one or more of the screenshots indicated in FIGS. 3A-3F, for example. In some cases, step 540, or any of the subsequent steps in FIG. 5B, may additionally, or alternatively, comprise broadcasting audible instructions to the driver/occupant.

As shown in FIG. 5B, in some cases, following (or before in other cases) initiation of a nystagmus test, preliminary diagnostics and/or actions may be performed at step 545. For example, in some implementations, a light, such as a specific light for the intoxication testing system that is configured to target a driver/occupant's face (such as light 416 in FIG. 4), or simply the internal lighting of the vehicle, may be triggered at step 545 to ensure that sufficient lighting is available in the cabin to accurately perform the nystagmus test. In some cases, the lighting may be tested for adequacy and then additional lighting actuated—or the user may be instructed to turn on additional lighting—in view of in indication of inadequate lighting.

In some cases, one or more preliminary tests may be performed at step 545 to confirm, or attempt to confirm, whether the subject being tested is the driver and/or the person matching the current user profile. For example, facial profiles, body profiles, and the like may be compared for a match with a current user profile.

Step 545 may also, or alternatively, be directed to detecting any spoofing or attempts to avoid intoxication detection that the user may attempt. For example, one or more cameras or other sensors may seek detection of any filters that may be present on or around the user's mouth or other objects that may interfere with accurate testing, such as sunglasses. Similarly, one or more cameras or other sensors may additionally, or alternatively, attempt to detect the presence of a second person within the seat and/or movements of the user and/or other persons in and/or out of the seat subject to the testing.

Similarly, step 545 may simply ensure the initial detection of the driver/occupant's pupils with adequate resolution and/or fidelity to proceed with testing.

If any of the preliminary diagnostics/testing fails, an error may be indicated at step 550. In some implementations, corrective instructions, such as “move closer to the camera/screen” or “please remove sunglasses” may then be provided at step 555. Similarly, some embodiments may be configured to, additionally or alternatively, initiate corrective actions at step 555. For example, if the lighting is insufficient, step 555 may comprise turning on and/or turning up the intensity of additional lighting.

If no errors are detected, the process may proceed to step 560 at which point the user may be instructed to stare at a dot or other fixed location displayed on a screen. One or more images of the driver/user's pupils may then be obtained and processed at step 565. If the pupil images are insufficient to complete the testing, a corrective instruction may be displayed and/or a corrective action taken at step 570. For example, a user may be instructed to open her eyes wider, remove glasses, or move closer to a camera/sensor. The sequence may then revert to step 560, or any other previous step, to restart the nystagmus test.

In some cases, initial intoxication testing may also be performed at step 565 using the pupil images. For example, the size of the test subject's pupils may be monitored and/or any movement of the pupils tracked for signs of nystagmus.

If the initial step has been performed without any indicia of problems, the process may proceed to step 575, at which point another instruction may be presented to the subject. For example, the subject may be instructed to move her head in a first direction, such as to one side, up, or down, preferably while maintaining eye contact with the fixed target on the display. In preferred embodiments, the instruction includes a dynamic graphical element to guide the subject's head in the desired direction. In some cases, this dynamic graphical element may be overlayed on the fixed target that moves relative to the fixed target in a direction and speed indicative of the desired direction and speed of movement of the subject's head during the nystagmus test.

Images taken at step 575 may then be processed at step 580 to detect signs of nystagmus. For example, the movement of the subject's pupils may be monitored for smoothness or lack thereof. In some cases, this may be done by comparing pupil locations in adjacent images in a sequence of images for movement. Movement in adjacent images by more than a threshold distance may be used to trigger a test “fail” or to provide an indicia of possible nystagmus and/or intoxication. In some cases, the degree to which the test results deviate from a normal test may be used to score or grade the test—e.g., to increase or decrease the likelihood of nystagmus and/or intoxication.

If the test is scored as a “fail,” the process may move to step 585, at which point the system may perform one or more actions, such as taking control of the vehicle, slowing/stopping the vehicle, preventing the vehicle from starting/moving, etc. Although not explicitly shown in the figure, in some cases, the test may be considered a “partial fail” and/or may be scored without a final result and continued with additional testing to confirm/deny nystagmus and/or intoxication. Alternatively, if the initial test is sufficiently definitive, the test may terminate at 585 in some cases.

If the initial test was negative and/or insufficiently definitive, the test may proceed to 590 for additional instructions. In some cases, however, the system may be configured to perform all of the instructions/tests in the full sequence every time, either irrespective of initial test results or because evaluation of the test results may not be completed until all data has been obtained. In other words, in some cases, method 500 may always proceed to step 590 and in others it need not, depending upon preliminary results of earlier steps/testing.

Step 590 may comprise displaying an instruction to hold the subject's head steady, preferably while continuing to stare at the fixed target, at the end of the movement in the first direction. For example, if the subject was instructed to move her head to the right in step 575, step 590 may comprise instructing the subject to hold her head steady while her head is rotated fully to the right. Again, preferably the user is further instructed to continue staring at the fixed target, which makes the test easier to perform and evaluate that having a target move all the way to the right (or another direction) while looking at it, as is typically done in field nystagmus testing.

The data obtained at step 590 may then be evaluated at step 595 to determine whether nystagmus has been detected and/or the likelihood/severity of nystagmus. As previously mentioned, however, in some cases the data obtained from each step of the test may be stored and evaluated together following the full test sequence instead of following each step. As with step 565, step 595 may comprise assessing any movement of the pupils while the subject attempts to hold them steady for signs of nystagmus. Again, if signs of nystagmus are present and/or are sufficiently compelling, in some cases the test may proceed to step 585.

If the test results are negative, inconclusive, or in embodiments in which a full test sequence is performed irrespective of preliminary results or in which preliminary results are not obtained, the test may proceed to step 600 at which point another instruction may be provided to the subject. In preferred implementations, this instruction may be to move the subject's head in a second direction, which may be in an opposite direction relative to the first direction. Thus, if the first direction was to the right, the second direction may be to the left. Similarly, if the first direction was up, the second direction may be down.

The results from step 600 may then be obtained and/or processed at step 605 to assess nystagmus and/or signs of nystagmus. Again, this step may comprise providing a score or other assessment of a likelihood of nystagmus in some cases rather than a binary score. Similarly, in some cases, the data from step 600 may simply be stored for later use in step 605.

In implementations in which the testing is to continue, another instruction may then be displayed at step 610. This step may, like step 590, be one to hold the subject's head steady, preferably while staring at the same fixed target, after the user has fully rotated/pivoted her head in the second direction.

The results from step 610 may then be obtained and/or processed at step 615 to assess nystagmus and/or signs of nystagmus. Again, this step may comprise providing a score or other assessment of a likelihood of nystagmus in some cases rather than a binary score. Similarly, in some cases, the data from step 610 may simply be stored for later use in step 615.

If all of the various tests have shown no signs of nystagmus, or if the signs of nystagmus have been insufficiently compelling and/or scored sufficiently low, the test may be considered passed at 620. In some cases, step 620 may further comprise providing a notification that the test has been passed and/or taking actions as a result of the test having been passed, such as releasing any restrictions on the vehicle being started or operated.

As used herein, a software module or component may include any type of computer instruction or computer executable code located within a memory device and/or m-readable storage medium. A software module may, for instance, comprise one or more physical or logical blocks of computer instructions, which may be organized as a routine, program, object, component, data structure, etc., that perform one or more tasks or implements particular abstract data types.

In certain embodiments, a particular software module may comprise disparate instructions stored in different locations of a memory device, which together implement the described functionality of the module. Indeed, a module may comprise a single instruction or many instructions, and may be distributed over several different code segments, among different programs, and across several memory devices. Some embodiments may be practiced in a distributed computing environment where tasks are performed by a remote processing device linked through a communications network. In a distributed computing environment, software modules may be located in local and/or remote memory storage devices. In addition, data being tied or rendered together in a database record may be resident in the same memory device, or across several memory devices, and may be linked together in fields of a record in a database across a network.

Furthermore, embodiments and implementations of the inventions disclosed herein may include various steps, which may be embodied in machine-executable instructions to be executed by a general-purpose or special-purpose computer (or another electronic device). Alternatively, the steps may be performed by hardware components that include specific logic for performing the steps, or by a combination of hardware, software, and/or firmware.

Embodiments and/or implementations may also be provided as a computer program product including a machine-readable storage medium having stored instructions thereon that may be used to program a computer (or other electronic device) to perform processes described herein. The machine-readable storage medium may include, but is not limited to: hard drives, floppy diskettes, optical disks, CD-ROMs, DVD-ROMs, ROMs, RAMS, EPROMS, EEPROMs, magnetic or optical cards, solid-state memory devices, or other types of medium/machine-readable medium suitable for storing electronic instructions. Memory and/or datastores may also be provided, which may comprise, in some cases, non-transitory machine-readable storage media containing executable program instructions configured for execution by a processor, controller/control unit, or the like.

The foregoing specification has been described with reference to various embodiments and implementations. However, one of ordinary skill in the art will appreciate that various modifications and changes can be made without departing from the scope of the present disclosure. For example, various operational steps, as well as components for carrying out operational steps, may be implemented in various ways depending upon the particular application or in consideration of any number of cost functions associated with the operation of the system. Accordingly, any one or more of the steps may be deleted, modified, or combined with other steps. Further, this disclosure is to be regarded in an illustrative rather than a restrictive sense, and all such modifications are intended to be included within the scope thereof. Likewise, benefits, other advantages, and solutions to problems have been described above with regard to various embodiments. However, benefits, advantages, solutions to problems, and any element(s) that may cause any benefit, advantage, or solution to occur or become more pronounced, are not to be construed as a critical, a required, or an essential feature or element.

Those having skill in the art will appreciate that many changes may be made to the details of the above-described embodiments without departing from the underlying principles of the inventions disclosed herein. The scope of the present inventions should, therefore, be determined only by the following claims.

1. A method for administration of an intoxication test sequence to a driver within a user interface of a vehicle, the method comprising the steps of:
displaying a fixed eye target on a graphical user interface of the vehicle;
displaying a first prompt on the graphical user interface inducing the driver to perform a first movement sequence in which the driver is to stare at the fixed eye target while moving the driver's head in a first direction;
receiving one or more images of the driver's eyes during the first movement sequence;
displaying a second prompt on the graphical user interface inducing the driver to perform a second movement sequence in which the driver is to stare at the fixed eye target while moving the driver's head in a second direction;
receiving one or more images of the driver's eyes during the second movement sequence;
processing received images of the driver's eyes to perform a nystagmus test; and
using the nystagmus test to assess whether the driver is intoxicated.

2. The method of claim 1, wherein the first prompt and the second prompt each comprises a dynamic graphical element that moves in a direction in which and a speed at which the driver is to move the driver's head during the first and second movement sequences.

3. The method of claim 2, wherein the dynamic graphical element pauses between the first and second movement sequences for a predetermined time period to indicate that the driver should hold the driver's head steady while staring at the fixed eye target for the predetermined time period.

4. The method of claim 2, wherein the dynamic graphical element is overlayed on the fixed eye target.

5. The method of claim 4, wherein the dynamic graphical element comprises an at least substantially spherical graphical element.

6. The method of claim 1, wherein the first prompt further comprises text prompting the user to perform the first movement sequence.

7. A system for intoxication testing within a vehicle, comprising:
an electromagnetic sensor configured to receive electromagnetic signals from an occupant of the vehicle;
a graphical display configured to administer an intoxication test sequence to the occupant of the vehicle, wherein the intoxication test sequence comprises a series of prompts to the occupant; and
an intoxication assessment module configured to assess potential intoxication of the occupant of the vehicle using electromagnetic signals received by the electromagnetic sensor during the intoxication text sequence.

8. The system of claim 7, wherein the graphical display comprises a graphical user interface.

9. The system of claim 7, wherein the intoxication assessment module comprises a nystagmus detection module, and wherein the intoxication test sequence comprises a nystagmus test sequence.

10. The system of claim 9, wherein the nystagmus test sequence comprises displaying a first prompt on the graphical display inducing the occupant to perform a first movement sequence in which the occupant is to stare at a fixed eye target displayed on the graphical display while moving the occupant's head in a first direction.

11. The system of claim 10, wherein the nystagmus test sequence further comprises displaying a second prompt on the graphical display inducing the occupant to perform a second movement sequence in which the occupant is to stare at the fixed eye target while moving the occupant's head in a second direction.

12. The system of claim 7, further comprising a breath sensor configured to detect an intoxicant level from breath of an occupant of a vehicle.

13. The system of claim 7, wherein the intoxication test sequence comprises a nystagmus test sequence.

14. The system of claim 13, wherein the series of prompts to the occupant comprises at least one prompt instructing the occupant to stare at an object displayed on the graphical display while moving the occupant's head.

15. A computing device comprising a display within a vehicle, the computing device being configured to:
display a first prompt to a driver of the vehicle indicating an initiation of an intoxication test, wherein the first prompt comprises a visual eye target for the driver;
display a second prompt to the driver to turn the driver's head in a first direction with the driver's eyes fixed on the visual eye target;
display a third prompt to the driver to turn the driver's head in a second direction with the driver's eyes fixed on the visual eye target; and
display a status indication to the driver as to the status of the intoxication test.

16. The computing device of claim 15, wherein the visual eye target does not move during the intoxication test.

17. The computing device of claim 15, wherein the first prompt further comprises a textual instruction indicating that the driver should stare directly at the visual eye target during the intoxication test.

18. The computing device of claim 15, wherein the second prompt comprises a dynamic graphical element that moves in the first direction to provide a visual cue to the driver to prompt movement of the driver's head in the first direction, and wherein the third prompt comprises a dynamic graphical element that moves in the second direction to provide a visual cue to the driver to prompt movement of the driver's head in the second direction.

19. The computing device of claim 18, wherein the dynamic graphical element of the second prompt and the dynamic graphical element of the third prompt each comprises a graphical element overlayed on the visual eye target that moves relative to the visual eye target in a direction and speed indicative of a preferred direction and speed of movement of the driver's head during the intoxication test.

20. The computing device of claim 15, wherein the computing device is further configured to:
display a first hold prompt in between the second prompt and the third prompt indicating a first time period during which the driver's head should be held steady before moving in the second direction; and
display a second hold prompt following the third prompt indicating a second time period during which the driver's head should be held steady following movement in the second direction.
